(19) United States
(12) Patent Application Publication
(10) Pub. No.: US 2023/0027059 A1
(43) Pub. Date: Jan. 26, 2023

(54) METHODS AND COMPOSITIONS FOR GENERATING HUMAN MIDBRAIN NEURAL PROGENITOR CELLS

(71) Applicant: Trallhead Biosystems Inc., Cleveland, OH (US)

(72) Inventor: Nooshin AMINI, Cleveland, OH (US)

(21) Appl. No.: 17/748,323

(22) Filed: May 19, 2022

Related U.S. Application Data
(60) Provisional application No. 63/223,139, filed on Jul. 19, 2021.

(51) Int. Cl. C12N 5/0793; C12N 5/0735; A61K 31/506; A61K 31/52; A61K 31/47; A61K 31/245; A61K 31/166

(52) U.S. Cl. CPC C12N 5/0619; C12N 5/0606; A61K 31/506; A61K 31/52; A61K 31/47; A61K 31/245; A61K 31/166; C12N 2506/02; C12N 2501/155; C12N 2501/115; C12N 2501/16; C12N 2501/415; C12N 2501/119; C12N 2501/727

(57) ABSTRACT

Methods for generating human committed midbrain neural stem cells (NSCs) and midbrain neural progenitor cells (midbrain NPCs) from human pluripotent stem cells are provided using chemically-defined culture media that allow for generation of the midbrain NPCs in as little as six days. The midbrain NPCs can be further differentiated to mature dopaminergic neurons. Culture media, isolated cell populations and kits are also provided.

### RELATED APPLICATIONS

This application claims priority to U.S. Provisional Application No. 63/223,139, filed Jul. 19, 2021, the entire contents of which is hereby incorporated by reference.

### GOVERNMENT LICENSED RIGHTS

This invention was made with government support under Grant Number: W911NF-17-3-0003 awarded by the U.S. ARMY ACC-AGP-RTP. The government has certain rights in the invention.

### BACKGROUND OF THE INVENTION

Parkinson's Disease (PD) is the second most common progressive neurodegenerative disease after Alzheimer's Disease and is characterized by degeneration of midbrain dopamine (mDA) neurons in the substantia nigra pars compacta. Current treatment typically takes a pharmacological approach aimed to increase dopamine bioavailability by administering levodopa (also known as L-dopa), the precursor to dopamine. However, side effects of long-term treatment with levodopa present challenges for its use in later stages of PD. The ability to reconstitute functional dopaminergic neurons in vivo in PD patients was first explored by transplanting human fetal midbrain tissue (reviewed in Lindvall et al. (2004) NeuroRx 1:383-393). Outcomes were variable and the approach raised ethical concerns about the availability and use of fetal tissue, leading to alternative approaches for reconstituting dopaminergic neurons in vivo.

The availability of pluripotent stem cells (PSCs), including embryonic stem (ES) cell lines and induced pluripotent stem cells (iPSCs), opened up the possibility of generating progenitors of mDA neurons in vitro. Developmental studies demonstrated that midbrain dopaminergic neurons originated from the ventral midbrain floor plate (mFP), which can be identified by co-expression of the markers FOXA2 and LMX1A. Early differentiation protocols for deriving midbrain floor plate precursors involved activation of sonic hedgehog (SHH) and canonical WNT signaling in PSCs, as well as dual SMAD inhibition and FGF8 activation, and required 11 days to achieve precursors expressing FOXA2 and LMX1A (Kriks et al. (2011) Nature 480:547-551) or involved activation of SHH, WNT and FGF8, as well as addition of retinoic acid (RA), for a 22-day protocol (Cooper et al. (2010) Mol. Cell. Neurosci. 45:258-266). A similar protocol has been reported in which human iPSCs-derived embryoid bodies were exposed to dual SMAD inhibition for five days, followed by SHH and FGF8 activation, leading to mDA precursors in 16 days (Hartfield et al. (2014) PLoS One 92:e87388).

More recently, additional protocols have been reported for obtaining MB dopaminergic progenitors from human pluripotent stem cells. For example, Nolbrant et al. report a 16 day protocol involving exposure to an N-2 supplement for the first 11 days and a B27 supplement for the last 5 days, as well as SHH and WNT activation and ALK inhibition (Nolbrant et al. (2017) Nature Protocols 12:1962-1979). Precious et al. report a protocol involving MEK inhibition for two days to block FGF signaling, followed by SHH activation alone for three days and SHH and FGF8 activation from day 5 onward, leading to FOXA2+LMX1A+progenitors by day 7 (Precious et al. (2020) Front. Neurosci. 14:312). Gartner et al. report a xeno-free, feeder-free chemically-defined protocol that involves incubation in media supplemented with (i) LDN193189 and SB431542 on days 0-5 and LDN193189 alone on days 5-10, (ii) CHIR99021 on days 2-13, and (iii) SHH and purmorphamine on days 1-7 (Gartner et al. (2020) Star Protocols 1:100065).

Human dopaminergic neuron progenitors have also been differentiated from human spermatogonial stem cells (hSSCs) using a protocol involving culture of the hSSCs for four days in olfactory ensheathing cell-conditioned medium (OECCM) supplemented with RA, SB, VPA and forskolin, followed by culture in OECCM supplemented with SHH, FGF8A and TFGβ3 (Yang et al. (2019) Stem Cell Res. Therap. 10:195).

Methods for expanding midbrain neural progenitor cells also have been described (Fedele et al. (2017) Sci. Reports 7:6036), as have methods of cryopreserving such progenitors (Drummond et al. (2020) Front. Cell. Dev. Biol. 8:578907).

Protocols for differentiating pluripotent stem cells into precursors of midbrain dopaminergic neurons are reviewed in, for example, Arenas et al. (2015) Development 142:1918-1936 and Wang et al. (2020) Cells 9:1489.

Accordingly, while some progress has been, there remains a need for efficient and robust methods and compositions for generating midbrain neural progenitor cells from human pluripotent stem cells.

### SUMMARY OF THE INVENTION

This disclosure provides methods of generating human committed midbrain (MB) neural stem cells (NSCs) and midbrain neural progenitor cells (NPCs) from pluripotent stem cells using chemically-defined culture media that allows for generation of OTX2+FOXA2+LMX1A+MB NPCs in as little as six days of culture. The culture media lacks serum or other exogenously-added growth factors and comprises small molecule agents that either agonize or antagonize particular signaling pathway activity in the pluripotent stem cells such that differentiation along the midbrain neural lineage is promoted, leading to cellular maturation and expression of midbrain neural progenitor-associated biomarkers. The methods of the disclosure have the advantage that use of small molecule agents in the culture media allows for precise control of the culture components and significantly shortens the differentiation time compared to prior art protocols.

Accordingly, in one aspect, the disclosure pertains to a method of generating human OTX2+FOXA2+LMX1A+midbrain neural progenitor cells (NPCs) comprising:

(a) culturing human pluripotent stem cells in a culture media lacking exogenously-added growth factors and comprising a WNT pathway agonist, an SHH pathway agonist, a BMP pathway antagonist, an AKT pathway antagonist and a MEK pathway antagonist on days 0-3 to obtain committed midbrain neural stem cells (NSCs); and

(b) culturing the committed midbrain NSCs in a culture media lacking exogenously-added growth factors and comprising a BMP pathway agonist, an RA pathway agonist, an LXR pathway agonist, an AKT pathway antagonist, an mTOR pathway antagonist and a TGFβ pathway antagonist on days 4-6 to obtain human OTX2+FOXA2+LMX1A+midbrain NPCs on day 6 of culture.

In another aspect, the disclosure pertains to a method of generating human OTX2+ LMX1A+committed midbrain neural stem cells (NSCs) comprising: culturing human pluripotent stem cells in a culture media lacking exogenously-added growth factors and comprising a WNT pathway agonist, an SHH pathway agonist, a BMP pathway antagonist, an AKT pathway antagonist and a MEK pathway antagonist on days 0-3 to obtain OTX2+LMX1A+committed midbrain NSCs on day 3 of culture.

Non-limiting examples of suitable agonist and antagonist agents, and concentrations therefor, for use in the methods of the disclosure are described in further detail herein. In one embodiment, the human pluripotent stem cells are induced pluripotent stem cells (iPSCs). In another embodiment, the human pluripotent stem cells are embryonic stem cells.

In on embodiment, the human pluripotent stem cells are attached to vitronectin-coated plates during culturing.

In another aspect, the disclosure pertains to a culture media for obtaining human committed midbrain neural stem cells comprising a WNT pathway agonist, an SHH pathway agonist, a BMP pathway antagonist, an AKT pathway antagonist and a MEK pathway antagonist and lacking exogenously-added growth factors.

In another aspect, the disclosure pertains to a culture media for obtaining human midbrain neural progenitor cells comprising a BMP pathway agonist, an RA pathway agonist, an LXR pathway agonist, an AKT pathway antagonist, an mTOR pathway antagonist and a TGFβ pathway antagonist, and lacking exogenously-added growth factors. I

In another aspect, the disclosure pertains to an isolated cell culture of human committed midbrain neural stem cells, the culture comprising: human OTX2+LMX1A+committed midbrain neural stem cells cultured in a culture media comprising a WNT pathway agonist, an SHH pathway agonist, a BMP pathway antagonist, an AKT pathway antagonist and a MEK pathway antagonist and lacking exogenously-added growth factors.

In another aspect, the disclosure pertains to an isolated cell culture of human midbrain neural progenitor cells, the culture comprising: human OTX2+FOXA2+LMX1A+midbrain neural progenitor cells cultured in a culture media comprising a BMP pathway agonist, an RA pathway agonist, an LXR pathway agonist, an AKT pathway antagonist, an mTOR pathway antagonist and a TGFβ pathway antagonist, and lacking exogenously-added growth factors.

In another aspect, the disclosure pertains to a human OTX2+FOXA2+LMX1A+midbrain neural progenitor cells generated by a method of the disclosure.

In another aspect, the disclosure pertains to a composition comprising a human midbrain neural progenitor cell (NPC), wherein the human midbrain NPC expresses OTX2, FOXA2 and LMX1A and lacks expression of GBX2.

In another aspect, the disclosure pertains to an isolated cell population of human midbrain neural progenitor cells (NPCs) comprising at least 1×10⁶ OTX2+FOXA2+LMX1A+human midbrain NPCs, wherein the cell population lacks GB X2-expressing neural stem cells. In an embodiment, the human midbrain NPCs are bound with at least one antibody that binds at least one marker expressed by the human midbrain NPCs.

Other features and advantages of the invention will be apparent from the following detailed description and claims.

### BRIEF DESCRIPTION OF THE DRAWINGS

FIG. 1 shows results from an HD-DoE model of a 13-factor experiment optimized for maximum expression of OTX2. The upper section of the model shows the prediction of expression level of pre-selected 53 genes when optimized for OTX2. The lower section of the model shows the effectors that were tested in this model and their contribution to maximum expression of OTX2. The value column refers to required concentration of each effector to mimic the model.

FIG. 2 shows the results from an HD-DoE model of a 13-factor experiment optimized for maximum expression of FOXA2. Upper and lower sections are as described for FIG. 1. This condition highlights the effector Purmorphamine with factor contribution of 22.2 as an important input for high expression of FOXA2.

FIG. 3 shows the dynamic profile of expression levels of OTX2, LMX1A, DMBX1 and FOXA2 genes relative to the concentration of 13 effectors tested. The positive impact of Purmorphamine, CHIR99021 and LDN193189 on expression of FOXA2 and their factor contribution is shown by the slope of the plots for each effector.

FIG. 4 shows the dynamic profile of expression levels of OTX2, LMX1A, DMBX1 and FOXA2 genes relative to the concentration of 13 effectors tested. The positive impact of MK2206, PD0325901, LDN193189 and CHIR99021 on expression of OTX2 and their factor contribution is shown by the slope of the plots for each effector.

FIG. 5 shows the results from an HD-DoE model of a 12-factor experiment applied on stage 1 neural stem cells to generate a recipe for stage 2 of differentiation. This model is optimized for maximum expression of LMX1A. This setting highlights the role of TTNPB in expression of LMX1A, with factor contribution of 19.5.

FIG. 6 shows the results from an HD-DoE model of a 12-factor experiment applied on stage 1 neural stem cells to generate a recipe for stage 2 of differentiation. This setting highlights the positive role of Purmorphamine and CHIR99021 on expression of FOXA2, with factor contribution of 15.3 and 10.7, respectively.

FIG. 7 shows the dynamic profile of expression levels of LMX1A, FOXA2 and GBX2 genes relative to the concentration of 12 effectors tested. The positive impact of TTNPB, CHIR99021 and GW3965 on expression of LMX1A and of Purmorphamine, MK2206 and GW3965 on the expression of FOXA2 and their factor contribution is shown by the slope of the plots for each effector.

FIG. 8 shows the results from an HD-DoE model of a 12-factor experiment applied on stage 1 neural stem cells to generate a recipe for stage 2 of differentiation. This setting highlights the positive role of BMP7 and MK2206 on expression of FOXA2, with factor contribution of 13.7 and 14.2, respectively.

FIG. 9 shows the results from an HD-DoE model of a 12-factor experiment applied on stage 1 neural stem cells to generate a recipe for stage 2 of differentiation. This setting highlights the positive role of MK2206 and the negative role of FGF8b on expression of LMX1A, with factor contribution of 12 and 16.9, respectively.

FIG. 10 shows the dynamic profile of expression levels of LMX1A, FOXA2 and GBX2 genes relative to the concentration of 12 effectors tested. The positive impact of BMP7 and MK2206 on expression of FOXA2 and of AZD 3147 on the expression of LMX1A and their factor contribution is shown by the slope of the plots for each effector.

FIGS. 11A-B shows the dynamic profile of expression levels of OTX2, DMBX1, FOXA2 and LMX1A genes relative to the concentration of 5 validated effectors tested in the recipe of stage 1 of differentiation. FIG. 11A shows the expression levels of genes of interest in the presence of all five finalized effectors. FIG. 11B shows the expression levels of genes of interest in the absence of one of the finalized effectors at a time when others area present.

FIGS. 12A-B shows the dynamic profile of expression levels of LMX1A, FOXA2 and GBX2 genes relative to the concentration of 3 validated effectors (TTNPB, A 83-01 and GW3965) tested in the recipe of stage 2 of differentiation. FIG. 12A shows the expression levels of genes of interest in the presence of all three finalized effectors. FIG. 12B shows the expression levels of genes of interest in the absence of one of the finalized effectors at a time when others area present.

FIGS. 13A-B shows the dynamic profile of expression levels of LMX1A, FOXA2 and GBX2 genes relative to the concentration of 3 validated effectors (MK2206, AZD 3147 and BMP7) tested in the recipe of stage 2 of differentiation. FIG. 13A shows the expression levels of genes of interest in the presence of all three finalized effectors. FIG. 13B shows the expression levels of genes of interest in the absence of one of the finalized effectors at a time when others area present.

FIG. 14 shows photographs of fluorescence images of MB-derived neural stem cells at the end of stage 1 treatment. Cells were stained with midbrain biomarkers including FOXA2, LMX1A, OTX2, mid-hindbrain boundary biomarker PAX2 and early neuronal biomarker Nestin and hindbrain biomarker GBX2. At this stage cells were positive for all the markers except FOXA2.

FIG. 15 shows photographs of fluorescence images of MB-derived neural stem cells at the end of stage 2 treatment. Cells were stained with midbrain biomarkers including FOXA2, LMX1A, OTX2 and hindbrain biomarker GBX2. At this stage cells were positive for all midbrain biomarkers and very low expression of GBX2 was observed.

FIG. 16 shows a schematic diagram of the two-stage culture protocol for generating midbrain neural progenitor cells from hiPCs in six days.

FIGS. 17A-C shows RNA-seq data of cells cultured in MB differentiation media after three days (stage 1) and six days (stage 2). FIG. 17A shows a bar plot of differential expression of selected genes at stage 1. At the end of stage 1, expression level of stem cell genes NANOG and POU5F1 were decreased while genes involved in early development of midbrain region and neuronal identity were elevated. FIG. 17B shows a bar plot of differential expression of selected genes at stage 2. At the end of stage 2, expression level of MB progenitor genes including DDC, LMX1B, SOX6 and EN1 increased. FIG. 17C shows a heatmap of gene profile of MB neural progenitors at day 6 compared to gene profile of hiPSCs at day 0.

### DETAILED DESCRIPTION OF THE INVENTION

Described herein are methodologies and compositions that allow for the generation of midbrain neural progenitors from human pluripotent stem cells under chemically-defined culture conditions using a small molecule based approach. The methods of the disclosure generate midbrain neural progenitors in a two stage protocol in which OTX2+LMX1A+committed MB neural stem cells (NSCs) are generated in three days, followed by generation of OTX2+ LMX1A+FOX2A+MB neural progenitor cells (NPCs) by day six of culture. Thus, the disclosure allows for obtention of MB NPCs in a significantly shorter time than prior art protocols using chemically-defined culture conditions.

As described in Example 1, a High-Dimensional Design of Experiments (HD-DoE) approach was used to simultaneously test multiple process inputs (e.g., small molecule agonists or antagonists) on output responses, such as gene expression. These experiments allowed for the identification of chemically-defined culture media, comprising agonists and/or antagonists of particular signaling pathways, that is sufficient to generate committed midbrain pluripotent stem cells and midbrain progenitor cells in a very short amount of time. The optimized culture media was further validated by a factor criticality analysis, which examined the effects of eliminating individual agonist or antagonist agents, as described in Example 2. Immunohistochemistry further confirmed the phenotype of the cells generated by the differentiation protocol, as described in Example 3. Furthermore, RNA-seq analysis of cells cultured according to the differentiation protocol also confirmed the expression of MB progenitor genes, as described in Example 4.

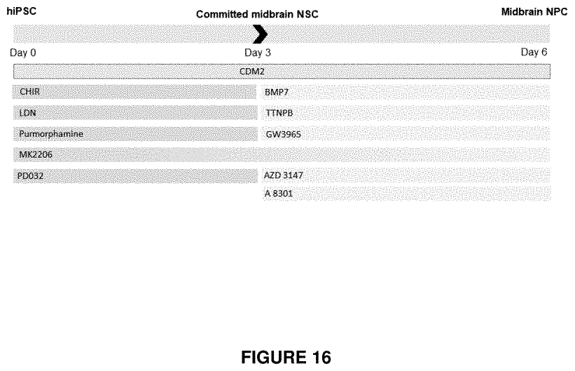

FIG. 16 schematically illustrates an embodiment of the method of the disclosure for generating MB NSCs and MB NPCs.

Various aspects of the invention are described in further detail in the following subsections.

### I. Cells

The starting cells used in the cultures of the disclosure are human pluripotent stem cells. As used herein, the term “human pluripotent stem cell” (abbreviated as hPSC) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can also evidenced by the expression of embryonic stem (ES) cell markers, although the preferred test for pluripotency is the demonstration of the capacity to differentiate into cells of each of the three germ layers.

Human pluripotent stem cells include, for example, induced pluripotent stem cells (iPSC) and human embryonic stem cells, such as ES cell lines. Non-limiting examples of induced pluripotent stem cells (iPSC) include 19-11-1, 19-9-7 or 6-9-9 cells (e.g, as described in Yu, J. et al. (2009) Science 324:797-801). Non-limiting examples of human embryonic stem cell lines include ES03 cells (WiCell Research Institute) and H9 cells (Thomson, J.A. et al. (1998) Science 282:1145-1147). Human pluripotent stem cells (PSCs) express cellular markers that can be used to identify cells as being PSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. Since the methods of generating committed midbrain neural stem cells and midbrain neural progenitor cells of the disclosure are used to differentiate (maturate) the starting pluripotent stem cell population, in various embodiments the midbrain-committed neural cell populations generated by the methods of the disclosure lack expression of one or more stem cell markers, such as one or more stem cell markers selected from the group consisting of TRA-1-60, TRA-1-81, TRA-2-54, SSEA1, SSEA3, SSEA4, CD9, CD24, OCT3, OCT4, NANOG and/or SOX2

The pluripotent stem cells are subjected to culture conditions, as described herein, that induce cellular differentiation. As used herein, the term “differentiation” refers to the development of a cell from a more primitive stage towards a more mature (i.e. less primitive) cell, typically exhibiting phenotypic features of commitment to a particular cellular lineage.

As used herein, a “neural stem cell” refers to a cell that is more differentiated than a pluripotent stem cell in that it is committed to the neural lineage but still has the capacity to differentiate into different types of cells along the neural lineage.

As used herein a “neural progenitor cell” refers to a cell that is more differentiated than a neural stem cell and that can be further differentiated into a particular type of neural cell.

In embodiments, cells can be identified and characterized based on expression of one or more biomarkers, such as particular biomarkers of neural progenitors or midbrain region-committed neural cells. Non-limiting examples of biomarkers whose expression can be assessed in the characterization of cells of interest include OTX2, which is a mesencephalic marker involved in positioning of midbrain and maintaining the mid-hindbrain boundary (Vernay et al. (2005) J. Neurosci. 25:4856-4867); LMX1A, which is involved in generation and differentiation of midbrain dopaminergic progenitors (Yan et al. (2011) J. Neurosci. 31:12413-12425); FOXA2, which regulates generation of midbrain dopaminergic neurons at early and late stages of development (Ferri et al. (2007) Development 134:2761-2769); PAX2, which is expressed in midbrain and anterior hindbrain (Urbanek et al. (1997) Proc. Natl. Acad. Sci. USA 94:5703-5708); Nestin, which is an early neuronal marker; KI67, which is a proliferation marker; and GBX2, which is a hindbrain marker.

As used herein, expression by a cell of only “low” levels of a biomarker of interest is intended to refer to a level that is at most 20%, and more preferably, less than 20%, less than 15%, less than 10% or less than 5% above background levels (wherein background levels correspond to, for example, the level of expression of a negative control marker that is considered to not be expressed by the cell).

In embodiments, the cells generated by the methods of the disclosure are committed midbrain (MB) neural stem cells (NSCs). As used herein, a “committed midbrain neural stem cell” or “committed MB NSC” refers to a stem cell-derived neural stem cell that expresses the biomarkers OTX2 and LMX1A. In an embodiment, the committed MB NSC does not express, or only expresses low levels of, the biomarker FOXA2. In an embodiment, the committed MB NSC does not express, or only expresses low levels of, the biomarker GBX2. In addition to OTX2 and LMX1A, a committed MB NSC may also express additional biomarkers, including but not limited to PAX2, Nestin and/or KI67.

In embodiments, the cells generated by the methods of the disclosure are midbrain neural progenitor cells, which are more differentiated (more mature) cells than committed MB NSCs. As used herein, a “midbrain neural progenitor cell” or “MB NPC” refers to a stem cell-derived progenitor cell that expresses the biomarkers OTX2, LMX1A and FOXA2. In an embodiment, the MB NPC does not express, or only expresses low levels of, the biomarker GBX2. In addition to OTX2, LMX1A and FOXA2, a MB NPC may also express additional biomarkers, including but not limited to PAX2, Nestin and/or KI67.

The committed MB NSCs and MB NPCs generated by the methods of the disclosure can be further cultured in vitro to generate mature dopaminergic neurons. Methods for differentiating midbrain neural progenitor cells to mature dopaminergic neurons are well established in the art, including protocols that involve further culture of the progenitor cells in medium containing BDNF, GDNF, ascorbic acid, DAPT and/or TGF-β.

### II. Culture Media Components

The methods of the disclosure for generating MB NSCs or MB NPCs comprise culturing human pluripotent stem cells in a culture media lacking exogenously-added growth factors and comprising specific agonist and/or antagonists of cellular signaling pathways.

As described in Example 1, a culture media comprising a WNT pathway agonist, an SHH pathway agonist, a BMP pathway antagonist, an AKT pathway antagonist and a MEK pathway antagonist was sufficient to generate OTX2- and LMX1A-expressing MB NSCs in as little as three days (referred to herein as “stage 1” of the differentiation protocol). Further differentiation of the MB NSCs in a culture media comprising a BMP pathway agonist, an RA pathway agonist, an LXR pathway agonist, an AKT pathway antagonist, an mTOR pathway antagonist and a TGF-β pathway antagonist was sufficient to generate OTX2+FOXA2+LMX1A+MB NPCs in another three days (referred to herein as “stage 2”), for an overall two-stage six day protocol.

As used herein, an “agonist” of a cellular signaling pathway is intended to refer to an agent that stimulates (upregulates) the cellular signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

Agonists and antagonists used in the methods of the disclosure are known in the art and commercially available. They are used in the culture media at a concentration effective to achieve the desired outcome, e.g., generation of midbrain NSCs and/or midbrain NPCs expressing midbrain markers of interest. Non-limiting examples of suitable agonist and antagonists agents, and effective concentration ranges, are described further below.

Agonists of the WNT pathway include agents, molecules, compounds, or substances capable of stimulating (upregulating) the canonical Wnt/β-catenin signaling pathway, which biologically is activated by binding of a Wnt-protein ligand to a Frizzled family receptor. In one embodiment, a WNT pathway agonist is a glycogen synthase kinase 3 (Gsk3) inhibitor. In one embodiment, the WNT pathway agonist is selected from the group consisting of CHIR99021, CHIR98014, SB 216763, SB 415286, LY2090314, 3F8, A 1070722, AR-A 014418, BIO, AZD1080, WNT3A, and combinations thereof. In one embodiment, the WNT pathway agonist is present in the culture media at a concentration within a range of 0.3-3.0 μM, 0.5-2.0 μM, 0.75-1.5 μM or 1.0-1.2 μM. In one embodiment, the WNT pathway agonist is CHIR99021. In one embodiment, the WNT pathway agonist is CHIR99021, which is present in the culture media at a concentration within a range of 0.3-3.0 μM, 0.5-2.0 μM, 0.75-1.5 μM or 1.0-1.2 04. In one embodiment, the WNT pathway agonist is CHIR99021, which is present in the culture media at a concentration of 1.1 04.

Agonists of the SHH (sonic hedgehog) pathway include agents, molecules, compounds, or substances capable of stimulating (activating) signaling through the SHH pathway, which biologically involves binding of SHH to the Patched-1 (PTCH1) receptor and transduction through the Smoothened (SMO) transmembrane protein. In one embodiment, the SHH pathway agonist is selected from the group consisting of Purmorphamine, GSA 10, SAG, and combinations thereof. In one embodiment, the SHH pathway agonist is present in the culture media at a concentration within a range of 100-1000 nM, 200-800 nM, 250-750 nM or 500-600 nM. In one embodiment, the SHH pathway agonist is Purmorphamine. In one embodiment, the SHH pathway agonist is Purmorphamine, which is present in the culture media at a concentration of 100-1000 nM, 200-800 nM, 250-750 nM or 500-600 nM. In one embodiment, the SHH pathway agonist is Purmorphamine, which is present in the culture media at a concentration of 550 nM.

Antagonists of the BMP (bone morphogenetic protein) pathway include agents, molecules, compounds, or substances capable of inhibiting (downregulating) the BMP signaling pathway, which biologically is activated by binding of BMP to a BMP receptor, which are activin receptor-like kinases (ALK) (e.g., type I BMP receptor, including but not limited to ALK2 and ALK3). In one embodiment, the BMP pathway antagonist is selected from the group consisting of LDN193189, DMH1, DMH2, Dorsomorphin, K02288, LDN214117, LDN212854, follistatin, ML347, Noggin, and combinations thereof. In one embodiment, the BMP pathway antagonist is present in the culture media at a concentration within a range of 100-500 nM, 100-400 nM, 150-350 nM or 200-300 nM. In one embodiment, the BMP pathway antagonist is LDN193189. In one embodiment, the BMP pathway antagonist is LDN193189, which is present in the culture media at a concentration within a range of 100-500 nM, 100-400 nM, 150-350 nM or 200-300 nM. In one embodiment, the BMP pathway antagonist is LDN193189, which is present in the culture media at a concentration of 275 nM.

Antagonists of the AKT pathway include agents, molecules, compounds, or substances capable of inhibiting (downregulating) the signaling pathway of one or more of the serine/threonine kinase AKT family members, which include AKT1 (also designated PKB or RacPK), AKT2 (also designated PKBβ or RacPK-β) and AKT 3 (also designated PKBγ or thyoma viral proto-oncogene 3). In one embodiment, the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, and combinations thereof. In one embodiment, the AKT pathway antagonist is present in the culture media at a concentration within a range of 25-300 nM, 50-250 nM, 75-200 nM or 125-150 nM. In one embodiment, the AKT pathway antagonist is MK2206. In one embodiment, the AKT pathway antagonist is MK2206, which is present in the culture media at a concentration within a range of 25-300 nM, 50-250 nM, 75-200 nM or 125-150 nM. In one embodiment, the AKT pathway antagonist is MK2206, which is present in the culture media at a concentration of 138 nM.

In an embodiment, the AKT pathway antagonist present in the culture media in step (a) is the same AKT pathway antagonist present in the culture media in step (b). In an embodiment, the AKT pathway antagonist present in the culture media in step (a) is a different AKT pathway antagonist than the AKT pathway antagonist present in the culture media in step (b). In an embodiment, the AKT pathway antagonist present in the culture media in both step (a) and step (b) is MK2206, e.g., which is present in the culture media in both steps at a concentration within a range of 25-300 nM, 50-250 nM, 75-200 nM or 125-150 nM, such as at 138 nM in both steps.

Antagonists of the MEK pathway include agents, molecules, compounds, or substances capable of inhibiting (downregulating) the signaling pathway of one or more of the components of the MAPK/ERK pathway (also known as the Ras-Raf-MEK-ERK pathway). In one embodiment, the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof. In one embodiment, the MEK pathway antagonist is present in the culture media at a concentration within a range of 25-300 nM, 50-250 nM, 75-200 nM or 100-120 nM. In one embodiment, the MEK pathway antagonist is PD0325901. In one embodiment, the MEK pathway antagonist is PD0325901, which is present in the culture media at a concentration within a range of 25-300 nM, 50-250 nM, 75-200 nM or 100-120 nM. In one embodiment, the MEK pathway antagonist is PD0325901, which is present in the culture media at a concentration of 110 nM.

Agonists of the RA pathway include agents, molecules, compounds, or substances capable of stimulation of a retinoic acid receptor (RAR) that is activated by both all-trans retinoic acid and 9-cis retinoic acid. There are three RARs: RAR-alpha, RAR-beta and RAR-gamma, which are encoded by the RARA, RARB, RARG genes, respectively. Different retinoic acid analogs have been synthesized that can activate the retinoic acid pathway. Non-limiting examples of such compounds include TTNPB (agonist of RAR-alpha, beta and gamma), AM 580 (RARalpha agonist), CD 1530 (potent and selective RARgamma agonist), CD 2314 (selective RARbeta agonist), Ch 55 (potent RAR agonist), BMS 753 (RARalpha-selective agonist), Tazarotene (receptor-selective retinoid; binds RAR-beta and -gamma), Isotretinoin (endogenous agonist for retinoic acid receptors; inducer of neuronal differentiation), and AC 261066 (RARβ2 agonist). In some embodiments, the RA signaling pathway agonist is selected from the group consisting of: i) a retinoid compound, ii) a retinoid X receptor (RXR) agonist, and iii) a 25 retinoic acid receptor (RARs) agonist. In particular embodiments, the RA pathway agonist is selected from the group consisting of: retinoic acid, Sr11237, adapalene, EC23, 9-cis retinoic acid, 13-cis retinoic acid, 4-oxo retinoic acid, and All-trans Retinoic Acid (ATRA).

Accordingly, in one embodiment, the RA pathway agonist is selected from the group consisting of TTNPB, AM 580, CD 1530, CD 2314, Ch 55, BMS 753, Tazarotene, Isotretinoin, AC 261066, retinoic acid (RA), Sr11237, adapalene, EC23, 9-cis retinoic acid, 13-cis retinoic acid, 4-oxo retinoic acid, and All-trans Retinoic Acid (ATRA), or combinations thereof. In one embodiment, the RA pathway agonist is present in the culture media at a concentration within a range of 5-500 nM, 25-250 nM, 10-100 nM or 25-75 nM. In one embodiment, the RA pathway agonist is TTNPB. In one embodiment, the RA pathway agonist is TTNPB, which is present in the culture media at a concentration within a range of 5-500 nM, 25-250 nM, 10-100 nM or 25-75 nM. In one embodiment, the RA pathway agonist is TTNPB, which is present in the culture media at a concentration of 50 nM.

Agonists of the LXR (liver X receptor) pathway include agents, molecules, compounds, or substances capable of stimulating (activating) signaling through the LXR pathway, which biologically involves heterodimerization of LXR with the retinoid X receptor (RXR) and activation by oxysterols. In one embodiment, the LXR pathway agonist is selected from the group consisting of GW3965, T0901317, DMHCA, AZ876, and combinations thereof. In one embodiment, the LXR pathway agonist is present in the culture media at a concentration within a range of 100-1000 nM, 200-800 nM, 250-750 nM or 550-650 nM. In one embodiment, the LXR pathway agonist is GW3965. In one embodiment, the LXR pathway agonist is GW3965, which is present in the culture media at a concentration of 100-1000 nM, 200-800 nM, 250-750 nM or 550-650 nM. In one embodiment, the LXR pathway agonist is GW3965, which is present in the culture media at a concentration of 500 nM.

Agonists of the BMP (bone morphogenetic protein) pathway include agents, molecules, compounds, or substances capable of stimulating (upregulating) the BMP signaling pathway, which biologically is activated by binding of BMP to a BMP receptor, which are activin receptor-like kinases (ALK) (e.g., type I BMP receptor, including but not limited to ALK2 and ALK3). In one embodiment, the BMP pathway agonist is selected from the group consisting of BMPs, sb4, ventromorphins (e.g., as described in Genthe et al. (2017) ACS Chem. Biol. 12:2436-2447), and combinations thereof. In one embodiment, the BMP pathway agonist is present in the culture media at a concentration within a range of 1-100 ng/ml, 5-50 ng/ml, 10-25 ng/ml or 12.5-17.5 ng/ml. In one embodiment, the BMP pathway agonist is BMP7. In one embodiment, the BMP pathway agonist is BMP7, which is present in the culture media at a concentration of 1-100 ng/ml, 5-50 ng/ml, 10-25 ng/ml or 12.5-17.5 ng/ml. In one embodiment, the BMP pathway agonist is BMP7, which is present in the culture media in step (b) of the method (i.e., stage 2) at a concentration of 15 ng/ml.

Antagonists of the TGFβ (transforming growth factor beta) pathway include agents, molecules, compounds, or substances capable of inhibiting (downregulating) signaling through a TGFβ receptor family member, a family of serine/threonine kinase receptors. In one embodiment, the TGFβ pathway antagonist is selected from the group consisting of A 83-01, SB-431542, GW788388, SB525334, TP0427736, RepSox, SD-208, and combinations thereof. In one embodiment, the TGFβ pathway antagonist is present in the culture media at a concentration within a range of 100-500 nM, 200-400 nM, 250-350 nM or 275-325 nM. In one embodiment, the TGFβ pathway antagonist is A 83-01. In one embodiment, the TGFβ pathway antagonist is A 83-01, which is present in the culture media at a concentration of 100-500 nM, 200-400 nM, 250-350 nM or 275-325 nM. In one embodiment, the TGFβ pathway antagonist is A 83-01, which is present in the culture media in step (b) of the method (i.e., stage 2) at a concentration of 300 nM.

Antagonists of the mTOR (mammalian target of rapamycin) pathway include agents, molecules, compounds, or substances capable of inhibiting (downregulating) signaling through mTOR, a PI3K-related kinase family member which is a core component of the mTORC1 and mTORC2 complexes. In one embodiment, the mTOR pathway antagonist is selected from the group consisting of AZD3147, rapamycin, sirolimus, temsirolimus, everolimus, ridaforolimus, umirolimus, zotarolimus, torin-1, torin-2, vistusertib, MHY1485, AZD8055, and combinations thereof. In one embodiment, the mTOR pathway antagonist is present in the culture media at a concentration within a range of 5-100 nM, 5-50 nM, 10-30 nM or 10-20 nM. In one embodiment, the mTOR pathway antagonist is AZD3147. In one embodiment, the mTOR pathway antagonist is AZD3147, which is present in the culture media at a concentration of 5-100 nM, 5-50 nM, 10-30 nM or 10-20 nM. In one embodiment, the mTOR pathway antagonist is AZD3147, which is present in the culture media in step (b) of the method (i.e., stage 2) at a concentration of 15 nM.

### III. Culture Conditions

In combination with the chemically-defined and optimized culture media described in subsection II above, the methods of generating committed MB NSCs and MB NPCs of the disclosure utilize standard culture conditions established in the art for cell culture. For example, cells can be cultured at 37 ° C. and under 5% O₂ and 5% CO₂ conditions. Cells can be cultured in standard culture vessels or plates, such as 96-well plates. In certain embodiments, the starting pluripotent stem cells are adhered to plates, preferably coated with an extracellular matrix material such as vitronectin. In one embodiment, the stem cells are cultured on a vitronectin coated culture surface (e.g., vitronectin coated 96-well plates).

Pluripotent stem cells can be cultured in commercially-available media prior to differentiation. For example, stem cells can be cultured for at least one day in Essential 8 Flex media (Thermo Fisher # A2858501) prior to the start of the differentiation protocol. In a non-limiting exemplary embodiment, stem cells are passaged onto vitronectin (Thermo Fisher # A14700) coated 96-well plates at 150,000 cells/cm2 density and cultured for one day in Essential 8 Flex media prior to differentiation.

To begin the differentiation protocol, the media the stem cells are being cultured in is changed to a basal differentiation media that has been supplemented with signaling pathway agonists and/or antagonists as described above in subsection II. A basal differentiation media can include, for example, a commercially-available base supplemented with additional standard culture media components needed to maintain cell viability and growth, but lacking serum (the basal differentiation media is a serum-free media) or any other exogenously-added growth factors, such as FGF2, PDGF, IGF or HGF. In a non-limiting exemplary embodiment, a basal differentiation media contains lx IMDM (Thermo Fisher # 12440046), 1×F12 (Thermo Fisher # 11765047), poly(vinyl alcohol) (Sigma # p8136) at 1 mg/ml, chemically defined lipid concentrate (Thermo Fisher # 11905031) at 1%, 1-thioglycerol (Sigma # M6145) at 450 uM,

Insulin (Sigma # 11376497001) at 0.7 ug/ml and transferrin (Sigma # 10652202001) at 15 ug/ml (also referred to herein as “CDM2” media, as used in the exemplary differentiation protocol shown in FIG. 16).

The culture media typically is changed regularly to fresh media. For example, in one embodiment, media is changed every 24 hours.

To generate committed MB NSCs and MB NPCs, the stem cells are cultured in the optimized culture media for sufficient time for cellular differentiation and expression of committed MB NSC- or MB NPC-associated markers. As described in the Examples, it has been discovered that culture of pluripotent stem cells in a two-stage method, one optimized for generation of MB NSCs and the other optimized for the generation of MB NPCs, can lead to the production of MB NPCs in as little as six days of culture, with the culture period for the first stage (leading to MB NSCs) being days 0-3 and the culture period for the second stage (leading to MB NPCs) being days 4-6.

Accordingly, in the first stage of the method, which generates MB NSCs, also referred to herein as “step (a)” or “stage 1”, pluripotent stem cells are cultured in the stage 1-optimized culture media on days 0-3, or starting on day 0 and continuing through day 3, or for 72 hours (3 days), or for at least 60 hours, or at least 64 hours, or at least 68 hours, or at least 70 hours, or at least 72 hours, or for 60 hours, or for 64 hours, or for 68 hours, or for 70 hours or for 72 hours.

Accordingly, in the second stage of the method, which generates MB NPCs, also referred to herein as “step (b)” or “stage 2”, the MB NSCs generated in step (a) are further cultured in the stage 2-optimized culture media on days 4-6, or starting on day 4 and continuing through day 6, or starting on day 4 and continuing for 72 hours (3 days), or starting on day 4 and continuing for at least 60 hours, or at least 64 hours, or at least 68 hours, or at least 70 hours, or at least 72 hours, or starting on day 4 and continuing for 60 hours, or for 64 hours, or for 68 hours, or for 70 hours or for 72 hours.

### IV. Uses

The methods and compositions of the disclosure for generating committed MB NSCs and MB NPCs allow for efficient and robust availability of these cell populations for a variety of uses. For example, the methods and compositions can be used in the study of midbrain neural progenitor development and biology, including differentiation into dopaminergic neurons, to assist in the understanding and potential treatment of neuronal diseases and disorders such as Parkinson's disease. For example, the committed MB NSCs and/or MB NPCs generated using the methods of the disclosure can be further purified according to methods established in the art using agents that bind to surface markers expressed on the cells. Accordingly, in one embodiment, the disclosure provides a method of isolating committed midbrain neural stem cells (committed MB NSCs) or midbrain neural progenitor cells (MB NPCs), the method comprising:

contacting MP NSCs or MP NPCs generated by a method of the disclosure with at least one binding agent that binds to a cell surface marker expressed by the MB NSCs or MB NPCs; and

isolating cells that bind to the binding agent to thereby isolate the MB NSCs or MB NPCs.

In one embodiment, the binding agent is an antibody, e.g., a monoclonal antibody (mAb) that binds to the cell surface marker. Cells that bind the antibody can be isolated by methods known in the art, including but not limited to fluorescent activated cell-sorting (FACS) and magnetic activated cell sorting (MACS).

Progenitors of the midbrain dopaminergic neural lineage also are contemplated for use in the treatment of neural diseases and disorders, through delivery of the cells to a subject having the disease or disorder, including but not limited to Parkinson's Disease.

### V. Compositions

In other aspects, the disclosure provides compositions related to the methods of generating committed MB NSCs and MB NPCs, including culture media and cell cultures, as well as isolated progenitor cells and cell populations.

In one aspect, the disclosure provides a culture media for obtaining human committed midbrain neural stem cells comprising a WNT pathway agonist, an SHH pathway agonist, a BMP pathway antagonist, an AKT pathway antagonist and a MEK pathway antagonist and lacking exogenously-added growth factors.

In another aspect, the disclosure provides a culture media for obtaining human midbrain neural progenitor cells comprising a BMP pathway agonist, an RA pathway agonist, an LXR pathway agonist, an AKT pathway antagonist, an mTOR pathway antagonist and a TGF-β pathway antagonist, and lacking exogenously-added growth factors.

In another aspect, the disclosure provides an isolated cell culture of human committed midbrain neural stem cells, the culture comprising: human OTX2+LMX1A+committed midbrain neural stem cells cultured in a culture media comprising a WNT pathway agonist, an SHH pathway agonist, a BMP pathway antagonist, an AKT pathway antagonist and a MEK pathway antagonist and lacking exogenously-added growth factors.

In another aspect, the disclosure provides an isolated cell culture of human midbrain neural progenitor cells, the culture comprising: human OTX2+FOXA2+LMX1A+midbrain neural progenitor cells cultured in a culture media comprising a BMP pathway agonist, an RA pathway agonist, an LXR pathway agonist, an AKT pathway antagonist, an mTOR pathway antagonist and a TGF-β pathway antagonist, and lacking exogenously-added growth factors.

In another aspect, the disclosure provides a human OTX2+FOXA2+LMX1A+midbrain neural progenitor cells generated by a method of the disclosure. In an embodiment, the disclosure pertains to a composition comprising a human midbrain neural progenitor cell (NPC), wherein the human midbrain NPC expresses OTX2, FOXA2 and LMX1A and lacks expression of, or has only low levels of expression of, GBX2. In an embodiment, the disclosure pertains to an isolated cell population of human midbrain neural progenitor cells (NPCs) comprising at least 1×10⁶ OTX2+FOXA2+LMX1A+human midbrain NPCs, wherein the cell population lacks GBX2-expressing neural stem cells. In an embodiment of the isolated cell population, the human midbrain NPCs are bound with at least one antibody that binds at least one marker expressed by the human midbrain NPCs.

The present invention is further illustrated by the following examples, which should not be construed as further limiting. The contents of figures and all references, patents and published patent applications cited throughout this application are expressly incorporated herein by reference.

### EXAMPLES

### Example 1

### Culture Protocol Development for the Generation of Stem Cell-Derived Midbrain Neural Progenitors Expressing FOXA2 and LMX1A

In this example, a two-stage culture protocol for generation of midbrain-derived neural progenitors was developed that can guide human pluripotent stem cells to progenitors expressing FOXA2 and LMX1A after 6 days in culture. These cells can be further differentiated to mature dopaminergic neurons.

This example utilizes a method of High-Dimensional Design of Experiments (HD-DoE), as previously described in Bukys et al. (2020) Iscience 23:101346. The method employs computerized design geometries to simultaneously test multiple process inputs and offers mathematical modeling of a deep effector/response space. The method allows for finding combinatorial signaling inputs that control a complex process, such as during cell differentiation.

It allows testing of multiple plausible critical process parameters, as such parameters impact output responses, such as gene expression. Because gene expression provides hallmark features of the phenotype of, for example, a human cell, the method can be applied to identify, and understand, which signaling pathways control cell fate. In the current example, the HD-DOE method was applied with the intent to find conditions for induction of midbrain neural progenitor-expressed genes, directly from the pluripotent stem cell state.

To develop the recipe for each stage, the impact of agonists and antagonists of multiple signaling pathways (referred to herein as “effectors”) on the expression of two sets of 53 pre-selected genes after a 3-day treatment was tested and modeled. These effectors are small molecules or proteins that are commonly used during stepwise differentiation of stem cells to specific fates. The choice of the effectors to test was based on current literature on neural induction in the midbrain region of the developing brain and differentiation of stem cells to neural progenitors.

To test the effectors, experiments with at least 8 factors were designed that can assess the response of cells to 48 or more different combinations of effectors in a range of concentrations. To analyze the models, we focused on expression of genes expressed in the midbrain region, including OTX2, DMBX1, FOXA2, LMX1A, and on the absence of GB X2, which is a hindbrain marker. The impact of each effector on gene expression level is defined by a parameter called factor contribution that is calculated for each effector during the modeling.

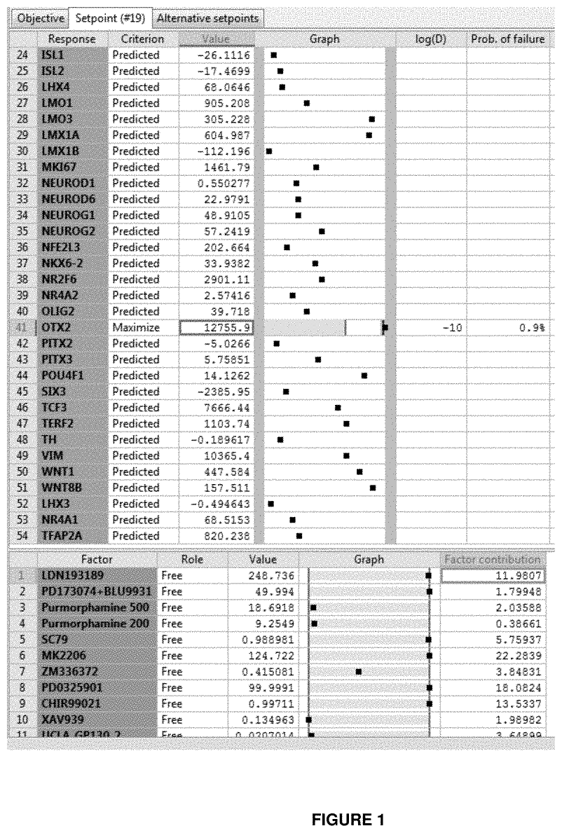

To identify the recipe of stage 1 of differentiation, cells were treated with various effectors for 3 days and the gene expression of cells was modeled. One model specifically, showed promising results on upregulation of DMBX1, LMX1A and OTX2 and downregulation of GBX2, when optimized for maximum expression of OTX2 at 12760.1. This model consisted of 13 factors including LDN193189, PD173074, BLU9931, Purmorphamine, SC79, MK2206, ZM336372, PD0325901, CHIR99021, XAV939, UCLA-gp130, Tofacitinib and GO 6983. Four of the effectors, MK2206 which is an antagonist of AKT signaling pathway, PD0325901 which is an antagonist of MEK signaling pathway, CHIR99021 which is an agonist of WNT signaling pathway and LDN193189 which is an antagonist of BMP signaling pathway had significant positive impact on expression of genes of interest with 22.3, 18.1, 13.5 and 11.9 factor contributions, respectively (FIG. 1).

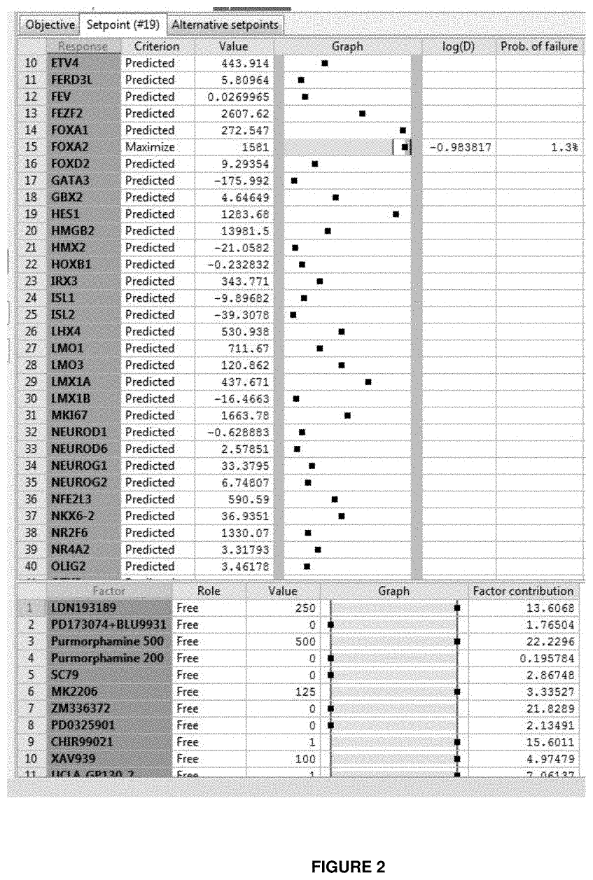

Since FOXA2 was not upregulated with optimization of OTX2, we next optimized the model for maximum expression of FOXA2 at 1581. Three effectors with significant positive effect on expression of FOXA2 were identified including LDN193189, CHIR99021 and Purmorphamine with 13.6, 15.6 and 22.2 factor contribution, respectively (FIG. 2). LDN193189 and CHIR99021 are common in both optimization settings and the rest of the factors except Purmorphamine, have a factor contribution less than 10. Therefore, we focused on impact of addition of Purmorphamine to the original 4 effectors.

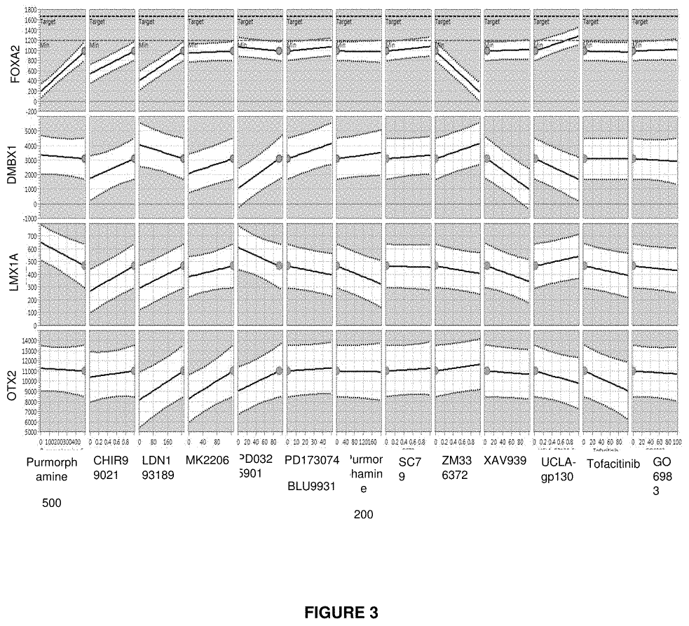

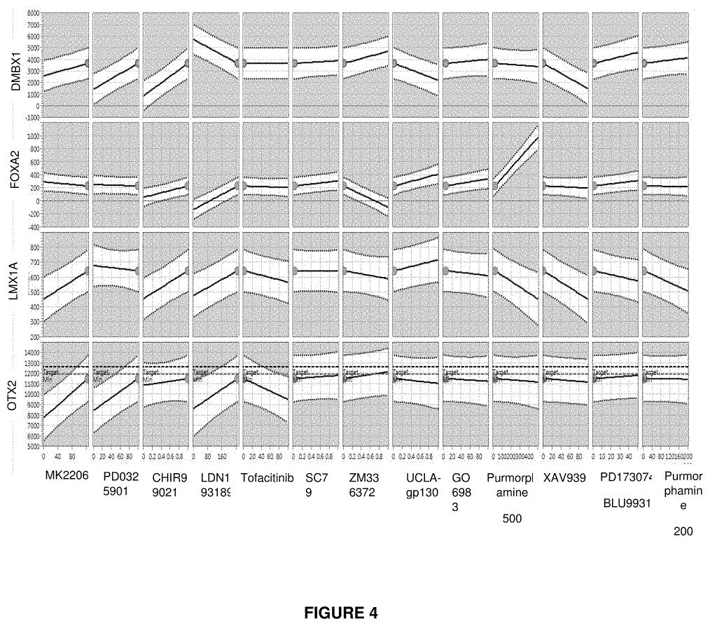

This assessment was done through dynamic profile analysis of the model with focus on expression of OTX2, DMBX1, LMX1A and FOXA2 (FIG. 3). Since Purmorphamine did not have a positive impact on expression of OTX2, DMBX1 and LMX1A in previous setting, we expected a drop in the expression level of the genes, however, it was observed that their expression level stayed in the same vicinity as the previous condition with 3000, 450 and 11000 for DMBX1, LMX1A and OTX2, respectively (FIG. 4).

The effectors validated for Stage 1 of the Protocol (generating midbrain committed neural stem cells) are summarized below in Table 1:

TABLE 1
Validated Effectors in Stage 1 of Protocol

| Effectors | Role | Concentration | |
|---|---|---|---|
| LDN193189 | BMP antagonist | 275 | nM |
| PD0325901 | MEK antagonist | 110 | nM |
| CHIR99021 | WNT agonist | 1.1 | uM |
| MK2206 | AKT antagonist | 138 | nM |
| Purmorphamine | SHH agonist | 550 | nM |

To further guide the differentiation of midbrain-committed neural stem cells to neural progenitor cells at stage 2, we performed an additional HD-DoE experiment. We thereby obtained additional gene regulatory models that were used for preparation of differentiation protocol. The basis of this was a 12-factor HD-DoE experiment with focus on initiation of differentiation of cells toward midbrain neural progenitor cells for an additional 3 days after termination of stage 1 treatment. Here, we focused on expression of LMX1A and FOXA2 in neural progenitor cells with low to zero expression of GBX2. LMX1A had significant high expression level in the model with value of 47888. Therefore, we used optimization setting for this gene to identify the positive factors. The factors in this experiment included SC79, MK2206, ZM336372, PD0325901, CHIR99021, A 83-01, TTNPB, AGN193109, GW3965, SR9243,

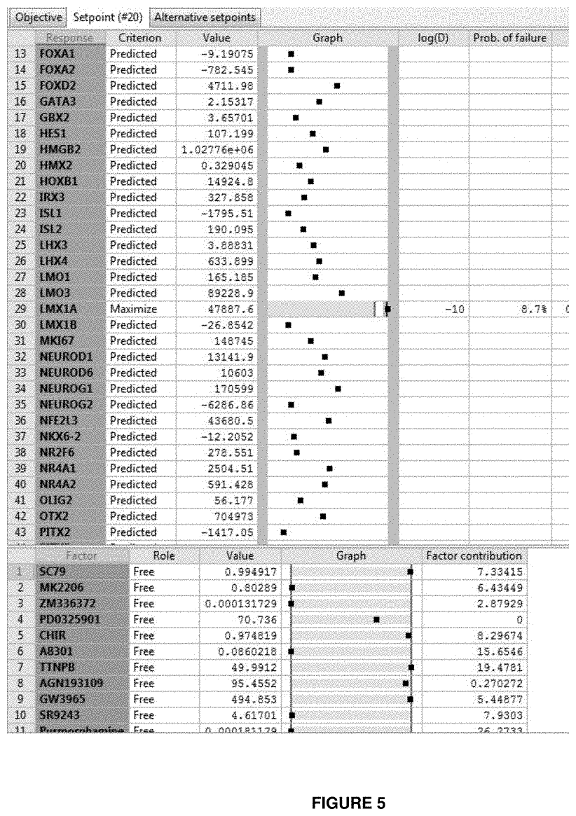

Purmorphamine and GSI-XX. When optimized for LMX1A, one factor, TTNPB, which is a small molecule agonist of RA signaling pathway, had significant positive impact with factor contribution of 19.5. CHIR99021, SC79 and GW3965 also had positive impacts but their factor contribution was less than 10 (8.3,7.3 and 5.4 respectively) and AGN193109 had <1 positive factor contribution (FIG. 5).

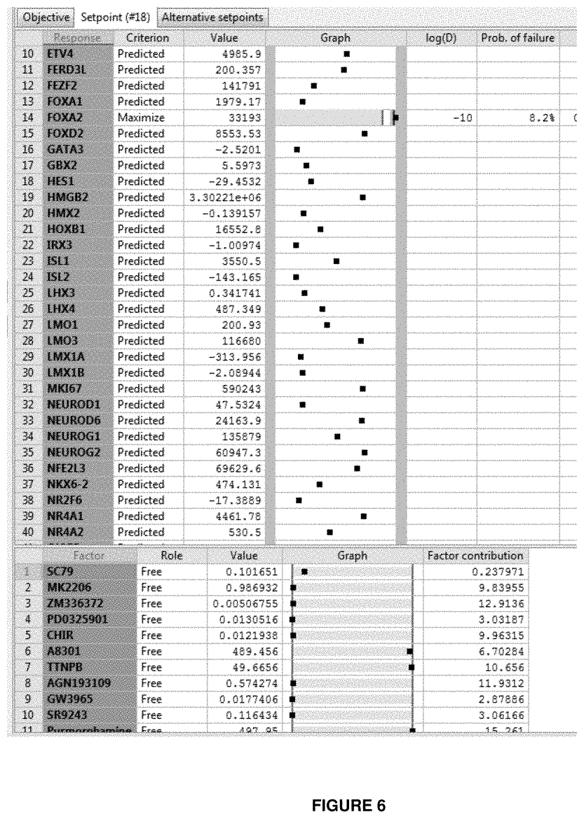

When the same experiment was optimized for maximum expression of FOXA2 at 33193, three effectors were identified with significant positive impact on expression of FOXA2 which include TTNPB, A 83-01 and Purmorphamine with 10.7, 6.7 and 15.3 factor contribution (FIG. 6).

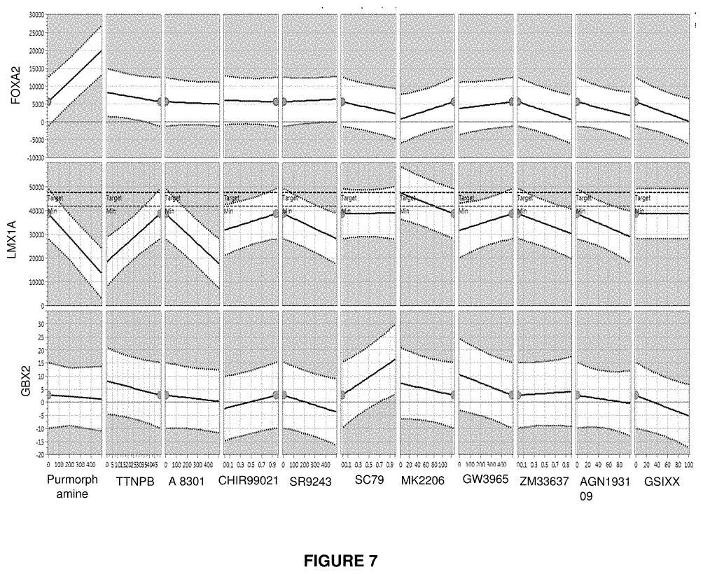

Similar to the experimental model of stage 1, the analysis again showed that Purmorphamine had a positive effect on FOXA2 expression levels and a negative effect on LMX1A expression levels, with factor contribution of 26.2. The model also revealed the same trend for A 83-01 and CHIR99021, with positive impacts only on FOXA2 and LMX1A, respectively. Therefore, we used dynamic profile analysis to adjust the recipe for optimized expression of both LMX1A and FOXA2 genes and minimum expression of GBX2 (FIG. 7). It was observed that even without addition of Purmorphamine, expression level of FOXA2 was at 5000, therefore inclusion of this effector is not essential, however it can improve expression of FOXA2. Based on dynamic profile plots, it was also concluded that even though CHIR99021 has positive effect on LMX1A, it also increases the expression level of GBX2 and reduces the relative expression of FOXA2 and therefore this factor was eliminated in the final recipe. A 83-01 was another factor with opposite effect on FOXA2 and LMX1A that was included in the final recipe. The dynamic profile analysis showed that addition of A 83-01 at a moderate concentration (300 nM) can improve expression of FOXA2 and help reducing the level of GBX2 to almost 0.

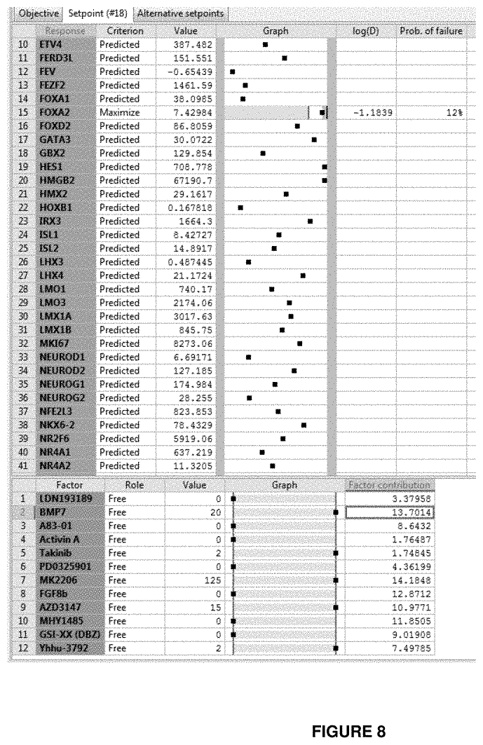

To test additional factors like FGF8 that is routinely used in midbrain differentiation protocols, we ran another 12-factor experiment consisting of LDN193189, BMP7, A 83-01, Activin A, Takinib, PD0325901, MK2206, FGF8b, AZD3147, MHY1485, GSI-XX and Yhhu 3792. Similar to the previous experiment, hiPSCs were treated with stage 1 media for 3 days and then treated with 96 conditions of combinations of the factors for an additional 3 days. When this model was optimized for maximum expression of FOXA2, BMP7 with factor contribution of 13.7 and MK2206 with 14.2 had the most impact on its expression followed by AZD 3147 with factor contribution of 10.9. Yhhu 3792 and takinib also had positive impacts but factor contributions were less than 10. Surprisingly, FGF8 had negative impact with factor contribution of 12.8 (FIG. 8).

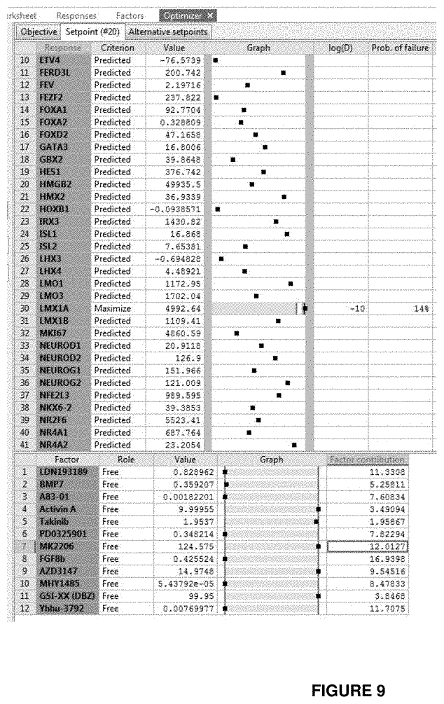

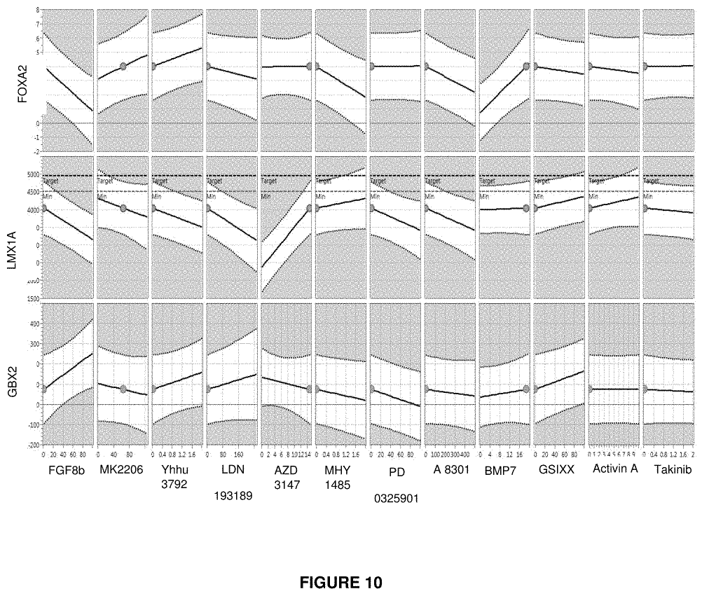

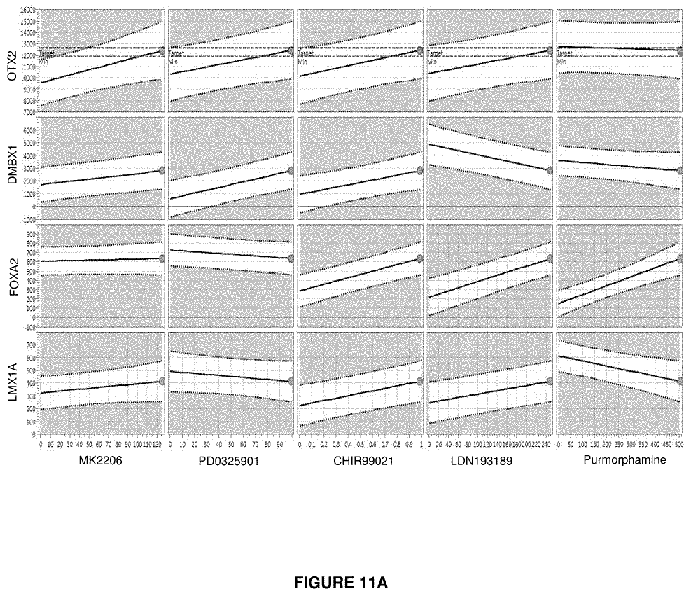

This model was also optimized for maximum expression of LMX1A, and MK2206 with factor contribution of 12 had the highest positive impact. AZD 3147, GSI-XX, Activin A and Takinib also had positive impact on its expression, but the factor contributions were less than 10 (FIG. 9). The model showed that Yhhu 3792 had a negative impact on expression of LMX1A, with factor contribution of 11.7, which is the opposite of FOXA2 condition. Another difference was BMP7, which has a negative impact on LMX1A. However, the factor contribution is less than 10. Therefore, to evaluate the effect of interactions between the factors and the optimal condition for the expression of both FOXA2 and LMX1A, we used dynamic profile analysis (FIG. 10).

Using dynamic profile analysis, we eliminated GSI-XX, Activin A and Takinib, since they did not make a meaningful positive change in the level of both FOXA2 and LMX1A expression. Yhhu 3792 was also eliminated since it had a considerable negative effect on LMX1A and positive effect on GBX2. BMP7 and AZD 3147 had significant positive impact on FOXA2 and LMX1A, respectively, while they did not reduce the expression of the other gene, therefore they were included in the final recipe. It was also shown that even though MK2206 decreases the level of LMX1A, it has desirable impact on FOXA2 and GBX2, therefore it was included in the final recipe at a moderate level.

The effectors validated for Stage 2 of the Protocol (generating midbrain-derived neural progenitor cells) are summarized below in Table 2:

TABLE 2
Validated Effectors in Stage 2 of Protocol

| Effectors | Role | Concentration | |
|---|---|---|---|
| TTNPB | RA agonist | 50 | nM |
| BMP7 | BMP pathway | 15 | ng/ml |
| GW3965 | LXR agonist | 500 | nM |
| A 83-01 | TGF-β antagonist | 300 | nM |
| AZD 3147 | mTOR antagonist | 15 | nM |
| MK2206 | AKT antagonist | 50 | nM |

Considering both models, conditions that maximize differentiation of cells to the midbrain region with neural progenitor cell identity as such relate to robust and elevated expression of OTX2, FOXA2 and LMX1A included the following effector inputs: TTNPB, BMP7, A 83-01, GW3965, AZD 3147 and MK2206.

The criticality of each individual validated effector for the stage 1 and stage 2 protocols was further evaluated as described in Example 2.

### Example 2

### Factor Criticality Analysis of Stem Cell Derived Midbrain Neural Progenitor-Inducing Culture Conditions

To assess the impact of elimination of each validated factor, we used dynamic profile analysis and compared the expression level of genes of interest in absence of each finalized factor while others are present. Since expression level of genes of interest reveal whether the desired outcome is reachable, this factor criticality analysis revealed the extent of importance of each input effector.

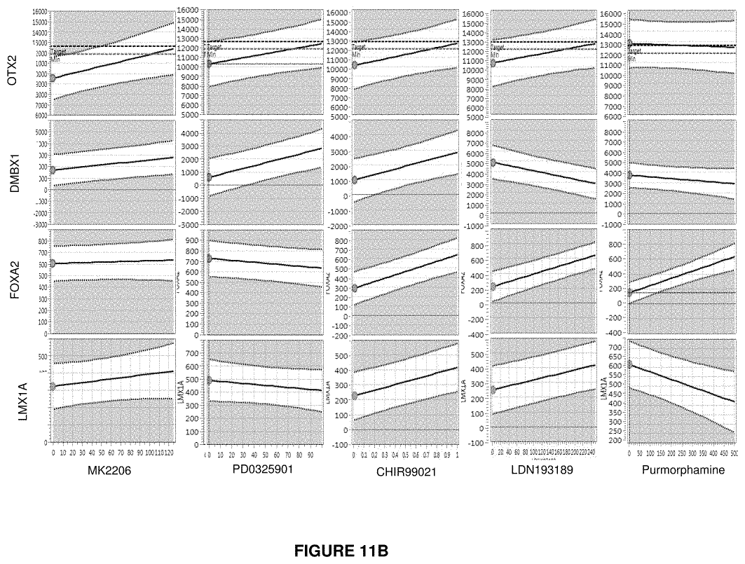

In the stage 1 recipe, each of the five finalized factors were removed while the other four factors were present and the expression levels of OTX2, DMBX1, FOXA2 and LMX1A was assessed compared to the levels in the presence of all five factors (FIGS. 11A-B). When MK2206 was removed, values of OTX2 and DMBX1 decreased from 12000 and 3000 to 9500 and 1500, respectively, while FOXA2 and LMX1A stayed the same. Absence of PD0325901 resulted in reduced expression of DMBX1 and it reached 900 while expression of FOXA2 and LMX1A increased from 600 and 300 to 700 and 500. Expression level of DMBX1 increased in absence of LDN193189, however values of OTX2, FOXA2 and LMX1A decreased. Values of all genes of interest was reduced after removing CHIR99021 which further proved its importance in stage 1 recipe and, as expected, absence of Purmorphamine lead to reduction of FOXA2 while it was beneficial for other genes.

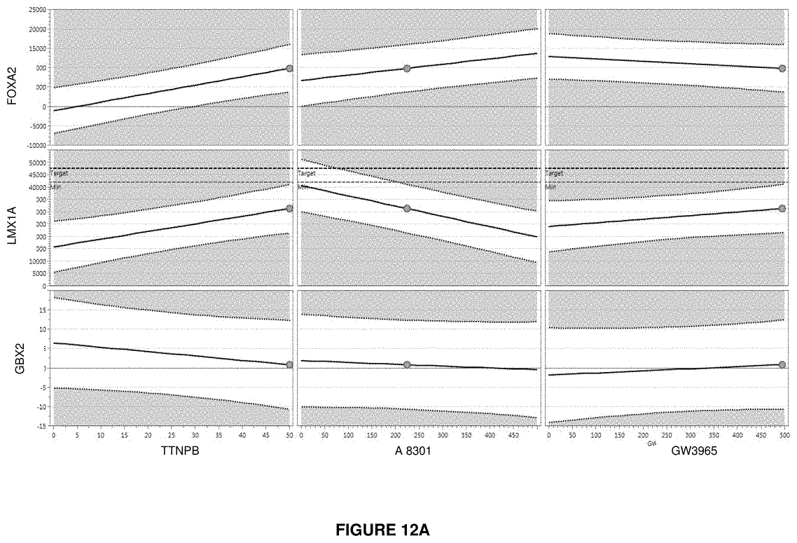

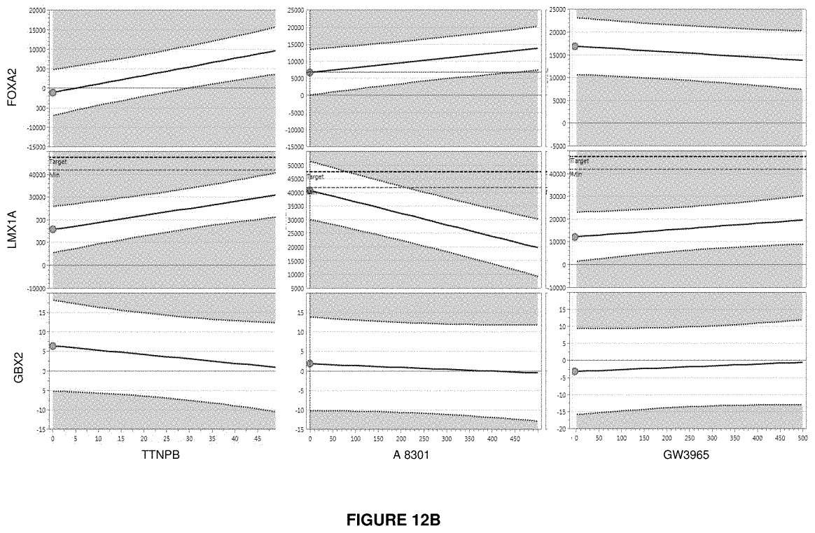

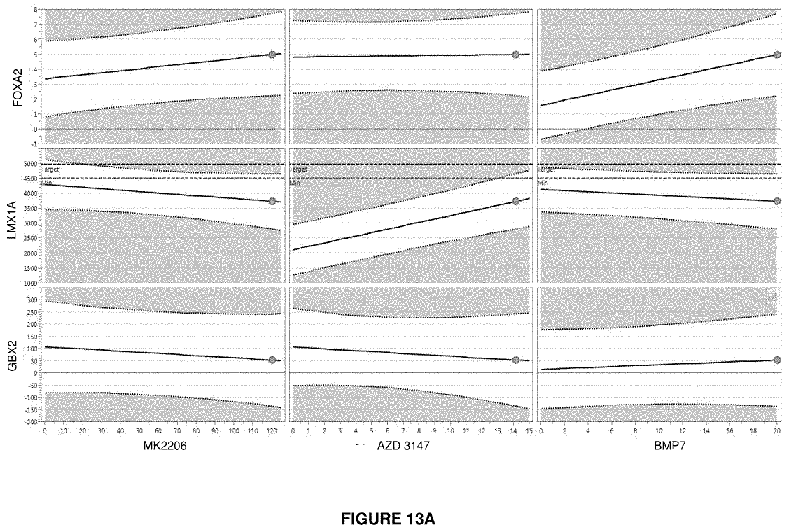

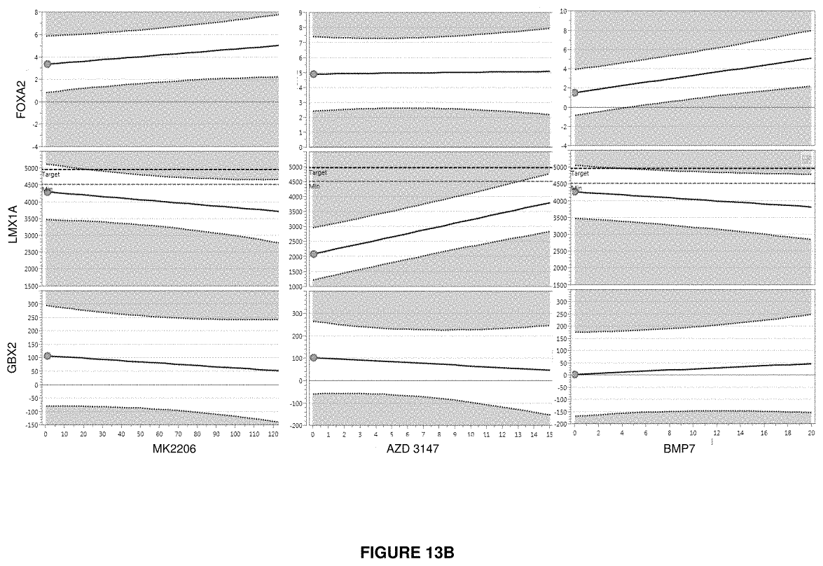

In the stage 2 recipe, each of the six finalized factors were removed while the other five factors were present and the expression levels of FOXA2, LMX1A and GBX2 were assessed, compared to the levels in the presence of all factors. According to the first experiment model, the absence of TTNPB lead to a rise of GBX2 expression, while the value of FOXA2 and LMX1A reduced drastically from 10,000 to 0 and 30,000 to 15,000, respectively. The absence of A 83-01 reduced the expression of FOXA2 from 10,000 to 7000, however, as expected, the value of LMX1A increased from 30,000 to 40,000. Deletion of GW3965 drastically reduced the value of LMX1A from 30,000 to 10,000 and increased the value of FOXA2 to 17,000 (FIGS. 12A-B). According to the second experiment model, the absence of BMP7 and MK2206 both reduced the level of FOXA2 while increasing the value of LMX1A, with BMP7 as the main effector that led to 0 expression of FOXA2. The absence of AZD 3147 reduced the level of LMX1A from 4000 to 2000 while having almost no effect on value of FOXA2 (FIGS. 13A-B) and therefore these factors were added to the final recipe.

### Example 3

### Immunocytochemistry Validation of Stem Cell-Derived Midbrain Neural Progenitors Expressing FOXA2 and LMX1A

To further validate the culture protocol developed as described in Example 1, cells were treated with stage 1 and stage 2 differentiation media, and immunocytochemistry was used to assess expression of biomarkers of the midbrain region and neural progenitors at the end of each stage. Biomarkers tested included OTX2, a mesencephalic marker involved in positioning of midbrain and maintaining the mid-hindbrain boundary (Vernay et al. (2005) J. Neurosci. 25:4856-4867), LMX1A which is involved in generation and differentiation of midbrain dopaminergic progenitors (Yan et al. (2011) J. Neurosci. 31:12413-12425), FOXA2, which regulates generation of midbrain dopaminergic neurons at early and late stages of development (Ferri et al. (2007) Development 134:2761-2769), PAX2, which is expressed in midbrain and anterior hindbrain (Urbanek et al. (1997) Proc. Natl. Acad. Sci. USA 94:5703-5708), Nestin, which is an early neuronal marker, KI67, which is a proliferation marker and GBX2, which is a hindbrain marker.

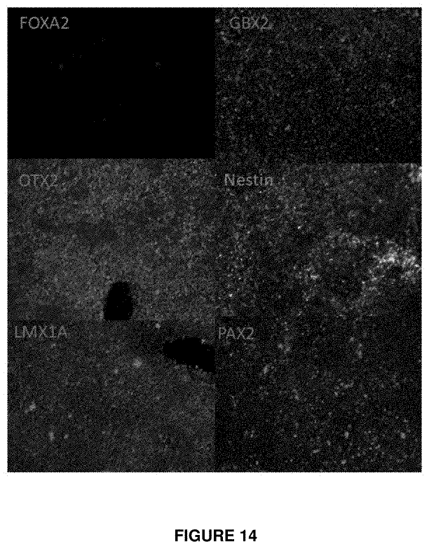

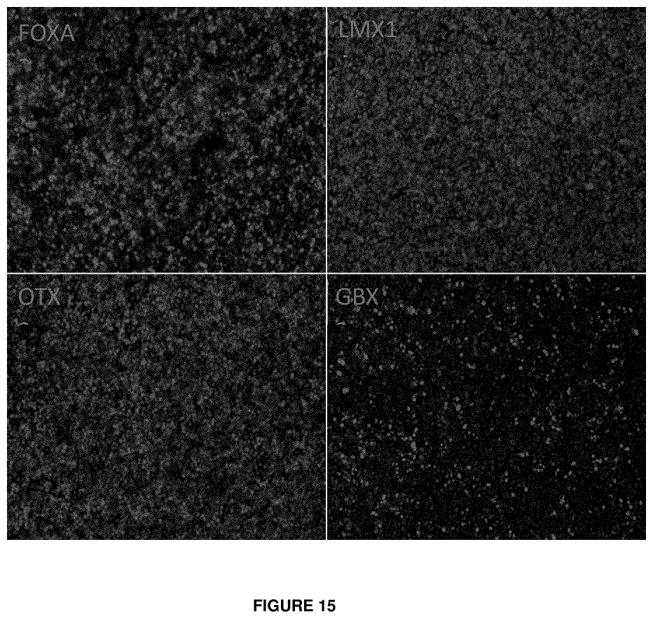

Immunocytochemistry images confirmed the expression of OTX2 and LMX1A in more than 90% of the cells by end of treatment with the stage 1 media. The markers PAX2, Nestin and KI67 were observed in some of the cells, as was some expression of GBX2. However, FOXA2 was not expressed (FIG. 14). After treatment with the stage 2 media, we observed that expression of LMX1A and OTX2 was maintained, while expression of FOXA2 was significantly increased, with detected of FOXA2 in more than 90% of the cells. GBX2 expression was also almost eliminated by end of stage 2 (FIG. 15). Detection of OTX2, LMX1A and FOXA2 by end of stage 2 of differentiation, while GBX2 was not detected, confirmed the recipes for stage 1 and stage 2 of differentiation of human induced pluripotent stem cells to midbrain neural progenitors after a 6-day treatment.

### Example 4

### RNA-seq Validation of Stem Cell-Derived Midbrain Neural Progenitors Expressing LMX1A and FOXA2

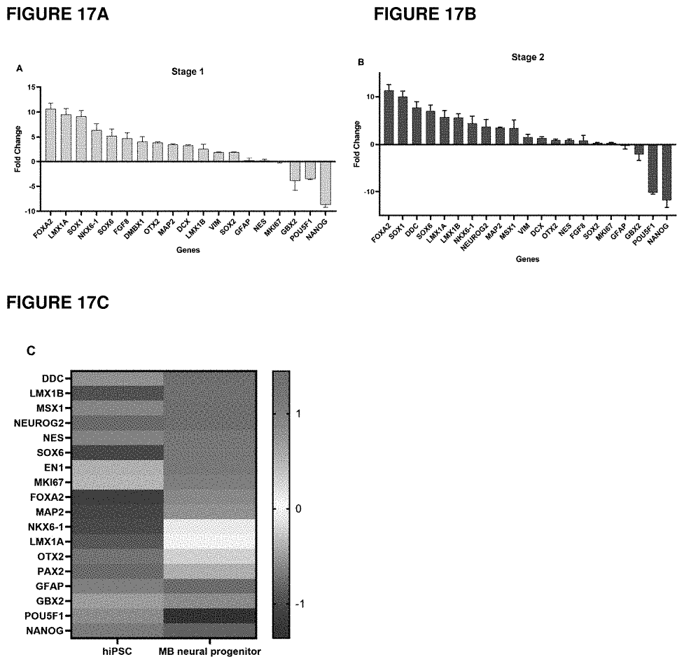

RNA sequencing was used to obtain the gene profile of cultured cells in the candidate recipes. Human iPSCs were cultured for a total of 6 days in stage 1 and 2 media and RNA of generated cells was sequenced at end of each stage. FIG. 17 shows normalized expression level of selected genes representative of midbrain region of the developing brain (OTX2, DMBX1, FOXA2, LMX1A), early neural identity (Nestin, SOX1, SOX2, Vimentin) and stem cell state (NANOG, POU5F1) in three replicates at day 0, day 3 and day 6. As it is shown in FIG. 17A and FIG. 17B, the level of stem cell genes has decreased in neural progenitors while the level of neuronal genes originating from midbrain region has increased; which validates the differentiation of hiPSCs to neural lineage with midbrain identity. FIG. 17A shows the fold change of 19 selected genes after 3 days treatment with stage 1 media compared to stage 0; FOXA2, LMX1A and SOX1 have the highest positive differential expression level (10.6, 9.5 and 9.1 respectively) compared to hiPSCs. Stemness genes NANOG and POU5F1, and also hindbrain gene GBX2, are at lowest levels (−8.7, -3.5 and -3.8 respectively). FIG. 17B shows the fold change of 21 selected genes of cells that were treated with stage 1 and stage 2 media compared to hiPSCs. FOXA2, SOX1 and DDC have the highest positive differential expression at 11.3, 10 and 7.7. Similar to stage 1, the lowest differential expression was observed with NANOG, POUF51 and GBX2. The heatmap of scaled gene profile of 18 selected genes of hiPSCs at day 0 and MB neural progenitors at day 6 (FIG. 17C) shows expression of midbrain progenitor genes, including DDC, LMX1A/B, SOX6, NEUROG2, FOXA2 and EN1, have increased after treatment with stage 1 and 2 media. The expression level of GFAP, a gene expressed in glial cells, was also observed and stayed the same during the 6-day differentiation, which shows the culture is mainly neuronal. This data demonstrates the ability of the stage 1 and stage 2 recipes as stage-wise differentiation media in directing the cells towards midbrain neuronal identity.

### EQUIVALENTS

Those skilled in the art will recognize, or be able to ascertain using no more than routine experimentation, many equivalents of the specific embodiments of the invention described herein. Such equivalents are intended to be encompassed by the following claims

1. A method of generating human OTX2+FOXA2+LMX1A+midbrain neural progenitor cells (NPCs) comprising:
(a) culturing human pluripotent stem cells in a culture media lacking exogenously-added growth factors and comprising a WNT pathway agonist, an SHH pathway agonist, a BMP pathway antagonist, an AKT pathway antagonist and a MEK pathway antagonist on days 0-3 to obtain committed midbrain neural stem cells (NSCs); and
(b) culturing the committed midbrain NSCs in a culture media lacking exogenously-added growth factors and comprising a BMP pathway agonist, an RA pathway agonist, an LXR pathway agonist, an AKT pathway antagonist, an mTOR pathway antagonist and a TGFβ pathway antagonist on days 4-6 to obtain human OTX2+FOXA2+LMX1A+midbrain NPCs on day 6 of culture.

2. The method of claim 1, wherein the human pluripotent stem cells are induced pluripotent stem cells (iPSCs).

3. The method of claim 1, wherein the human pluripotent stem cells are embryonic stem cells.

4. The method of claims 1, wherein the human pluripotent stem cells are attached to vitronectin-coated plates during culturing.

5. The method of claim 1, wherein the WNT pathway agonist is selected from the group consisting of CHIR99021, CHIR98014, SB 216763, SB 415286, LY2090314, 3F8, A 1070722, AR-A 014418, BIO, AZD1080, WNT3A and combinations thereof.

6. The method of claim 5, wherein the WNT pathway agonist is present in the culture media at a concentration within a range of 0.5-2.0 μM.

7. The method of claim 5, wherein the WNT pathway agonist is CHIR99021, which is present in the culture media at a concentration of 1.1 μM in steps (a) and (b).

8. The method of claim 1, wherein the SHH pathway agonist is selected from the group consisting of Purmorphamine, GSA 10, SAG, and combinations thereof.

9. The method of claim 8, wherein the SHH pathway agonist is present in the culture media at a concentration within a range of 200-800 nM.

10. The method of claim 8, wherein the SHH pathway agonist is Purmorphamine, which is present in the culture media at a concentration of 550 nM.

11. The method of claim 1, wherein the BMP pathway antagonist is selected from the group consisting of LDN193189, DMH1, DMH2, Dorsopmorphin, K02288, LDN214117, LDN212854, folistatin, ML347, Noggin, and combinations thereof.

12. The method of claim 11, wherein the BMP pathway antagonist is present in the culture media at a concentration within a range of 100-500 nM.

13. The method of claim 11, wherein the BMP pathway antagonist is LDN193189, which is present in the culture media at a concentration of 275 nM.

14. The method of claim 1, wherein the AKT pathway antagonist is selected from the group consisting of MK2206, GSK690693, Perifosine (KRX-0401), Ipatasertib (GDC-0068), Capivasertib (AZD5363), PF-04691502, AT 7867, Triciribine (NSC154020), ARQ751, Miransertib (ab235550), Borussertib, Cerisertib, and combinations thereof.

15. The method of claim 14, wherein the AKT pathway antagonist is present in the culture media at a concentration within a range of 25-300 nM.

16. The method of claim 14, wherein the AKT pathway antagonist is MK2206, which is present in the culture media at a concentration of 138 nM in step (a) and 50 nM in step (b).

17. The method of claim 1, wherein the MEK pathway antagonist is selected from the group consisting of PD0325901, Binimetinib (MEK162), Cobimetinib (XL518), Selumetinib, Trametinib (GSK1120212), CI-1040 (PD-184352), Refametinib, ARRY-142886 (AZD-6244), PD98059, U0126, BI-847325, RO 5126766, and combinations thereof.

18. The method of claim 17, wherein the MEK pathway antagonist is present in the culture media at a concentration within a range of 25-300 nM.

19. The method of claim 17, wherein the MEK pathway antagonist is PD0325901, which is present in the culture media at a concentration of 110 nM.

20. The method of claim 1, wherein the BMP pathway agonist is selected from the group consisting of BMPs, sb4, ventromorphins, and combinations thereof.

21. The method of claim 20, wherein the BMP pathway agonist is present in the culture media at a concentration within a range of 10-25 ng/ml.

22. The method of claim 20, wherein the BMP pathway agonist is BMP7, which is present in the culture media at a concentration of 15 ng/ml.

23. The method of claim 1, wherein the RA pathway agonist is selected from the group consisting of TTNPB, AM 580, CD 1530, CD 2314, Ch 55, BMS 753, Tazarotene, Isotretinoin, AC 261066, retinoic acid (RA), Sr11237, adapalene, EC23, 9-cis retinoic acid, 13-cis retinoic acid, 4-oxo retinoic acid, All-trans Retinoic Acid (ATRA), and combinations thereof.

24. The method of claim 23, wherein the RA pathway agonist is present in the culture media at a concentration within a range of 10-100 nM.

25. The method of claim 23, wherein the RA pathway agonist is TTNPB, which is present in the culture media at a concentration of 50 nM.

26. The method of claim 1, wherein the LXR pathway agonist is selected from the group consisting of GW3965, T0901317, DMHCA, AZ876, and combinations thereof.

27. The method of claim 26, wherein the LXR pathway agonist is present in the culture media at a concentration within a range of 250-750 nM.

28. The method of claim 26, wherein the LXR pathway agonist is GW3965, which is present in the culture media at a concentration of 500 nM.

29. The method of claim 1, wherein the mTOR pathway antagonist is selected from the group consisting of AZD3147, rapamycin, sirolimus, temsirolimus, everolimus, ridaforolimus, umirolimus, zotarolimus, torin-1, torin-2, vistusertib, MHY1485, AZD8055, and combinations thereof.

30. The method of claim 29, wherein the mTOR pathway antagonist is present in the culture media at a concentration within a range of 10-30 nM.

31. The method of claim 29, wherein the mTOR pathway antagonist is AZD3147, which is present in the culture media at a concentration of 15 nM.

32. The method of claim 1, wherein the TGFβ pathway antagonist is selected from the group consisting of A 83-01, SB-431542, GW788388, SB525334, TP0427736, RepSox, SD-208, and combinations thereof.

33. The method of claim 32, wherein the TGFβ pathway antagonist is present in the culture media at a concentration of 100-500 nM.

34. The method of claim 32, wherein the TGFβ pathway antagonist is A 83-01, which is present in the culture media in step (b) at a concentration of 300 nM.

35. A method of generating human OTX2+LMX1A+committed midbrain neural stem cells (NSCs) comprising:
culturing human pluripotent stem cells in a culture media lacking exogenously-added growth factors and comprising a WNT pathway agonist, an SHH pathway agonist, a BMP pathway antagonist, an AKT pathway antagonist and a MEK pathway antagonist on days 0-3 to obtain OTX2+LMX1A+committed midbrain NSCs on day 3 of culture.

36. A culture media for obtaining human committed midbrain neural stem cells comprising a WNT pathway agonist, an SHH pathway agonist, a BMP pathway antagonist, an AKT pathway antagonist and a MEK pathway antagonist and lacking exogenously-added growth factors.

37. A culture media for obtaining human midbrain neural progenitor cells comprising a BMP pathway agonist, an RA pathway agonist, an LXR pathway agonist, an AKT pathway antagonist, an mTOR pathway antagonist and a TGFβ pathway antagonist, and lacking exogenously-added growth factors.

38. An isolated cell culture of human committed midbrain neural stem cells, the culture comprising: human OTX2+LMX1A+committed midbrain neural stem cells cultured in a culture media comprising a WNT pathway agonist, an SHH pathway agonist, a BMP pathway antagonist, an AKT pathway antagonist and a MEK pathway antagonist and lacking exogenously-added growth factors.

39. An isolated cell culture of human midbrain neural progenitor cells, the culture comprising:
human OTX2+FOXA2+LMX1A+midbrain neural progenitor cells cultured in a culture media comprising a BMP pathway agonist, an RA pathway agonist, an LXR pathway agonist, an AKT pathway antagonist, an mTOR pathway antagonist and a TGFβ pathway antagonist, and lacking exogenously-added growth factors.

40. A human OTX2+FOXA2+LMX1A+midbrain neural progenitor cells generated by the method of claim 1.

41. A composition comprising a human midbrain neural progenitor cell (NPC), wherein the human midbrain NPC expresses OTX2, FOXA2 and LMX1A and lacks expression of GBX2.

42. An isolated cell population of human midbrain neural progenitor cells (NPCs) comprising at least 1×10⁶ OTX2+FOXA2+LMX1A+human midbrain NPCs, wherein the cell population lacks GBX2-expressing neural stem cells.

43. The isolated cell population of claim 42, wherein the human midbrain NPCs are bound with at least one antibody that binds at least one marker expressed by the human midbrain NPCs.
